(19) United States
(12) Patent Application Publication
(10) Pub. No.: US 2026/0108215 A1
(43) Pub. Date: Apr. 23, 2026

(54) METHOD, DEVICE, AND COMPUTER PROGRAM FOR PREDICTING PROGNOSIS OF INTRAVITREAL INJECTION THERAPY

(71) Applicant: SAMSUNG LIFE PUBLIC WELFARE FOUNDATION, Seoul (KR)

(72) Inventors: Je Moon YOON, Seoul (KR); Hakje YOO, Seoul (KR); Myung Jin CHUNG, Seoul (KR); Sung Ho JUNG, Seoul (KR)

(21) Appl. No.: 19/365,913

(22) Filed: Oct. 22, 2025

(30) Foreign Application Priority Data
Oct. 22, 2024 (KR) 10-2024-0144687

(51) Int. Cl. A61B 5/00; A61B 3/10; G06T 7/00; G06T 7/11; G06T 7/174; G16H 20/17

(52) U.S. Cl. CPC A61B 5/7275; A61B 3/102; G06T 7/0014; G06T 7/11; G06T 7/174; G16H 20/17; G06T 2207/10101; G06T 2207/20081; G06T 2207/20084; G06T 2207/30041; G06T 2207/30096

(57) ABSTRACT

The present invention relates to a method for predicting the prognosis of intravitreal injection treatment, comprising the steps of: receiving OCT images and clinical information of a plurality of diabetic macular edema patients; extracting one or more major lesion information from the OCT images; training a multi-modal deep learning model by inputting the OCT images, the clinical information, and the one or more major lesion features, and outputting the intravitreal injection treatment prognosis of the diabetic macular edema patients; and predicting the prognosis of intravitreal injection treatment by inputting the OCT images, the clinical information, and the major lesion features of a specific diabetic macular edema patient to the trained multi-modal deep learning model.

### CROSS-REFERENCE TO RELATED APPLICATIONS

The present application is based on and claims priority to Korean Patent Application No. KR10-2024-0144687 filed on Oct. 22, 2024, the entire contents of which are hereby incorporated by reference.

### TECHNICAL FIELD

The present invention relates to a method, a device, and a computer program for predicting a prognosis of intravitreal injection therapy, and more particularly, to a method, a device, and a computer program for predicting a prognosis of intravitreal injection therapy using clinical information of a diabetic macular edema patient and an optical coherence tomography (OCT) image.

### DESCRIPTION OF THE RELATED ART

Diabetic macular edema (DME) is a major cause of vision impairment that can occur in all stages of diabetic retinopathy. A 10-year longitudinal study showed that diabetic macular edema occurred in 20% of patients with type 1 diabetes and 25% of patients with type 2 diabetes. As the prevalence of type 2 diabetes increases worldwide and the life expectancy of diabetic patients is prolonged, it is estimated that more than 20 million people worldwide are affected by diabetic macular edema.

Currently, an intravitreal injection is the main treatment for diabetic macular edema. If the response to this treatment is insufficient, other treatment options such as laser treatment and surgery should be considered. Therefore, predicting the response to intravitreal injection therapy is very important in determining the appropriate options.

Various features of OCT (Optical coherence tomography) images and systematic factors related to treatment response or prognosis of diabetic macular edema after intravitreal injection have been reported.

Recently, attempts have been made to predict the therapeutic response to intravitreal injection using artificial intelligence technology. However, existing studies often perform AI learning using only OCT images without considering systematic factors, include only formulations that can be used only in a specific area, or fail to achieve a satisfactory level of accuracy in predicting treatment response.

### RELATED ART DOCUMENT

U.S. Pat. No. 10,943,348

### SUMMARY OF INVENTION

### Problems to be Solved

An object of the present invention is to provide a method, a device, and a computer program for predicting a prognosis of intravitreal injection therapy capable of predicting a treatment response to intravitreal injection using a multi-modal deep learning model trained with OCT images, extracted features of diabetic macular edema lesions, and clinical data of a patient.

### Means for Solving Problems

In order to achieve the above object, the present invention provides a method for predicting a prognosis of intravitreal injection treatment, the method comprising the steps of: receiving OCT images and clinical information of a plurality of diabetic macular edema patients; extracting one or more major lesion information from the OCT images; training a multi-modal deep learning model by inputting the OCT images, the clinical information, and the one or more major lesion features, and outputting the intravitreal injection treatment prognosis of the diabetic macular edema patients; and predicting the prognosis of intravitreal injection treatment by inputting the OCT images, clinical information, and the major lesion features of a specific diabetic macular edema patient to the trained multi-modal deep learning model.

Preferably, the extracting of the main lesion information may include extracting five slices of two sheets on each side based on a slice of the center of the macula from the OCT image, extracting a region of interest (ROI) corresponding to the center of the macula from the extracted five slices using a retinal region mask, and extracting the main lesion information in the ROI using a main lesion mask.

Preferably, the main lesion information may include at least one of the location, size, number, and distribution of the diabetic macular edema lesion.

Preferably, the clinical information may include at least one of age, sex, type of diabetes, glycated hemoglobin level, presence or absence of hypertension, presence or absence of hyperlipidemia, lens condition, vitrectomy history, and previous treatment history of the patient.

Preferably, the multi-modal deep learning model may have a structure in which a Convolutional Neural Network (CNN) for extracting features of an OCT image and a Deep Neural Network (DNN) for extracting features of clinical information and main lesion information are combined.

Preferably, the predicting of the intravitreal injection treatment prognosis may include inputting the OCT image to a convolutional neural network to extract a first feature, inputting the clinical information and the main lesion information to a deep neural network to extract a second feature, combining the first feature and the second feature in a late-fusion manner, and inputting the combined first feature and second feature to a fully-connected network (FCN) to predict the intravitreal injection treatment prognosis of a diabetic macular edema patient.

Preferably, in the predicting of the prognosis of intravitreal injection treatment, the prognosis may be predicted based on the amount of change in central retinal thickness after one or more intravitreal injection treatments.

In addition, the present invention is a device for predicting the prognosis of intravitreal injection therapy, comprising: a processor including one or more cores; and a memory, wherein the processor is configured to receive OCT images and clinical information of a plurality of diabetic macular edema patients, extract one or more main lesion information from the OCT images, input the OCT images, the clinical information, and the one or more main lesion features, and train a multi-modal deep learning model by outputting the intravitreal injection treatment prognosis of the diabetic macular edema patients.

In addition, the present invention is a computer program including instructions stored in a computer-readable storage medium to cause a computer to perform the following operations, wherein the operations include: receiving an OCT image and clinical information of a plurality of diabetic macular edema patients; extracting one or more main lesion information from the OCT image; training a multi-modal deep learning model by inputting the OCT image, the clinical information, and the one or more main lesion features, and outputting an intravitreal injection treatment prognosis of a diabetic macular edema patient; and predicting an intravitreal injection treatment prognosis by inputting the OCT image, clinical information, and the main lesion features of a specific diabetic macular edema patient to the trained multi-modal deep learning model.

### Effects of Invention

The present invention has an advantage in that it is possible to predict a patient's response or prognosis to intravitreal injection treatment, which is a popular treatment for diabetic macular edema, through a multiple modal prognosis prediction model, thereby securing evidence information on the treatment effect and treatment plan of the patient.

### BRIEF DESCRIPTION OF DRAWINGS

FIG. 1 is a flowchart of a method of predicting a prognosis of intravitreal injection treatment according to an embodiment of the present invention;

FIG. 2 is a schematic diagram of a method for predicting a prognosis of intravitreal injection treatment according to an embodiment of the present invention;

FIG. 3 illustrates an OCT image preprocessing process performed in a step of extracting major lesion information according to an embodiment of the present invention;

FIG. 4 is a performance index of a CNN-based model according to a first embodiment of the present invention;

FIG. 5 shows the feature importance of the top 20 combinations according to the first embodiment of the present invention;

FIG. 6 shows a performance index of a CNN-based model according to a second embodiment of the present invention;

FIG. 7 shows the feature importance of the top 20 combinations according to the second embodiment of the present invention;

FIG. 8 is a configuration diagram of a device for predicting a prognosis of intravitreal injection treatment according to an embodiment of the present invention; and

FIG. 9 is a schematic diagram of a computing environment according to an embodiment of the present invention.

### DETAILS FOR CARRYING OUT THE INVENTION

Hereinafter, the present invention will be described in detail with reference to the contents described in the accompanying drawings. However, the present disclosure is not limited by the exemplary embodiments. Like reference numerals presented in each drawing denote members that perform substantially the same function.

The objects and effects of the present disclosure may be naturally understood or become clearer by the following description, and the objects and effects of the present disclosure are not limited only by the following description. In addition, in describing the present disclosure, when it is determined that the detailed description of the known technology related to the present disclosure may unnecessarily obscure the gist of the present disclosure, the detailed description thereof will be omitted.

The terms used in the present invention are used only to describe specific embodiments, and are not intended to limit the present invention. Singular expressions include plural expressions unless the context clearly indicates otherwise. In the present application, it is to be understood that the terms such as “include” or “have” are intended to designate the presence of features, numbers, steps, operations, components, parts, or a combination thereof described in the description of the invention, and do not preclude the possibility of presence or addition of one or more other features, numbers, steps, operations, components, parts, or a combination thereof.

Terms such as first, second, etc. may be used to describe various components, but the components should not be limited by the terms. The terms are used only for the purpose of distinguishing one component from another. For example, without departing from the scope of the present disclosure, a first component may be referred to as a second component, and similarly, a second component may also be referred to as a first component.

Unless otherwise defined, all terms used herein, including technical or scientific terms, have the same meaning as those generally understood by those of ordinary skill in the art to which the present invention pertains. Terms such as those defined in commonly used dictionaries should be interpreted as having a meaning consistent with the meaning in the context of the related art, and unless clearly defined in the present invention, they are not interpreted as ideal or excessively formal meanings.

In interpreting the components, it is interpreted as including the error range even if there is no separate explicit description. The description of the temporal relationship includes, for example, a case in which the temporal precedence relationship is described as ‘after’, ‘following’, ‘subsequently’, ‘then’, ‘before’, etc., and a case in which the temporal precedence relationship is not continuous unless ‘immediately’ or ‘directly’ is used.

Hereinafter, the technical configuration of the present invention will be described in detail with reference to the accompanying drawings.

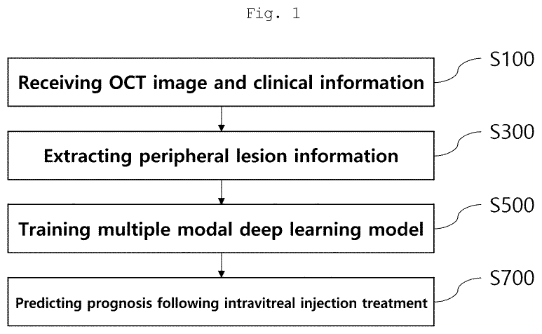

FIG. 1 is a flowchart of a method of predicting a prognosis of intravitreal injection treatment according to an embodiment of the present invention. Referring to FIG. 1, a method of predicting a prognosis of intravitreal injection treatment may include receiving an OCT image and clinical information (S100), extracting peripheral lesion information (S300), training a multi-modal deep learning model (S500), and predicting a prognosis of intravitreal injection treatment (S700).

The method of predicting the prognosis of intravitreal injection treatment may use a multi-modal deep learning model that combines OCT images and clinical information to predict the prognosis of intravitreal injection treatment in diabetic macular edema patients. Here, the intravitreal injection treatment may include anti-vascular endothelial growth factor (anti-VGEF) injection treatment and steroid injection treatment.

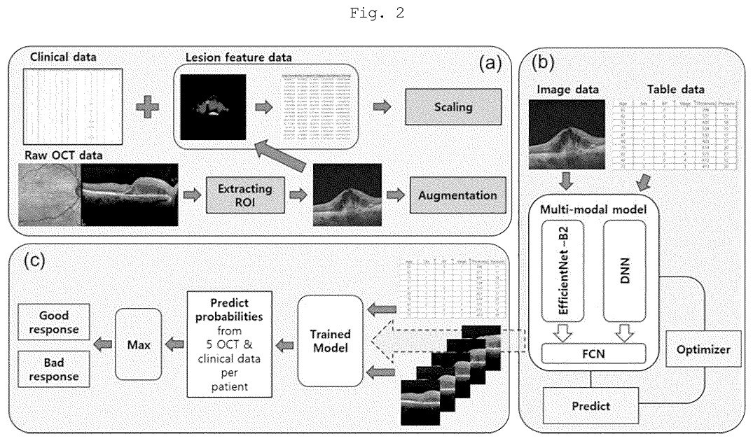

FIG. 2 is a schematic diagram of a method for predicting a prognosis of intravitreal injection treatment according to an embodiment of the present invention. Referring to FIG. 2A, the method of predicting the prognosis of intravitreal injection treatment may include a data preprocessing process for training a multi-modal deep learning model, and the data preprocessing process may include an OCT image processing process, a main lesion information extraction process, and a clinical information filtering process.

Referring to FIG. 2B, the method of predicting the prognosis of intravitreal injection treatment may obtain weights by performing single modal learning with a model suitable for each data type (OCT image, clinical information, and major lesion information). In this case, the obtained weight may be used as it is in the feature extractor of the multi-modal deep learning model. Finally, the multi-modal deep learning model may train a fully convolutional networks (FCN) layer after fusion of features extracted from a single modal model.

Referring to FIG. 2C, the method of predicting the prognosis of intravitreal injection treatment may evaluate the prediction performance of the trained model, obtain a prediction probability using five OCT images for each patient and table data matching the same, and obtain a prediction value based on a maximum value among the prediction probabilities.

In the step S100 of receiving the OCT image and clinical information, the OCT image and clinical information of a plurality of diabetic macular edema patients may be received.

The OCT image and clinical information may be used to train a multi-modal deep learning model. The OCT image and clinical information may be obtained from patients who have received one or more consecutive intravitreal injection treatments at one month intervals among diabetic macular edema patients. Preferably, the anti-VEGF therapeutic agent may be a patient who has received three intravitreal injection treatments, and the steroid treatment agent may be a patient who has received one intravitreal injection treatment.

The OCT image may be captured through the Spectral domain-optical coherence tomography. The OCT image may be taken in 31 or 19 horizontal B-scan images from one patient.

The clinical information may include at least any one of age, sex, diabetes type, glycated hemoglobin level, presence or absence of high blood pressure, presence or absence of hyperlipidemia, lens condition, vitrectomy history, and previous treatment history of the patient.

The clinical information may include information on a patient's history such as age, sex, high blood pressure, hyperlipidemia, type of diabetes, glycated hemoglobin, lens condition, vitrectomy history, injection and laser treatment history, and OCT central subfield thickness information measured before and after treatment. The basic information and history may be collected at the time of the first visit, and the examination information may be measured immediately after treatment for each round and after more than one month.

In the step S300 of extracting the peripheral lesion information, one or more pieces of main lesion information may be extracted from the OCT image.

The main lesion information may include at least one of the location, size, number, and distribution of the diabetic macular edema lesion.

In the step S300 of extracting the peripheral lesion information, the information collected in the step S100 of receiving the OCT image and clinical information may be preprocessed. The extracting of the peripheral lesion information (S300) may exclude examination information one month after the first intravitreal injection treatment and examination information measured immediately after the second and third treatments and one month after the first treatment from the clinical information (it is determined that the examination information immediately after the first treatment is the same as the result of the examination immediately before the treatment and is used for learning).

The step of extracting the peripheral lesion information (S300) may be excluded from the training data when there is a missing value for the history information. The step of extracting the peripheral lesion information (S300) is not replaced by applying a preprocessing technique such as an interpolation method to the patient's unique history. In an embodiment, in the extracting of the peripheral lesion information (S300), in the case of a patient treated with the anti-VEGF therapeutic agent, the central retinal thickness immediately after the treatment and the central retinal thickness after the third treatment are compared, and if the central retinal thickness is reduced by 50 μm or more, the prognosis may be classified as “Good response”, and if the central retinal thickness is less than or increased, the prognosis may be classified as “Bad response”. In another embodiment, in the extracting of the peripheral lesion information (S300), in the case of a patient treated with a steroid therapeutic agent, in the case of a male, the central retinal thickness after the first treatment is compared, and when the initial thickness is 320 to 374 μm, the central retinal thickness is reduced by 50 μm or more, when the initial thickness is 375 to 494 μm, the central retinal thickness is reduced by 100 μm or more, and when the initial thickness is 495 μm or more, the central retinal thickness is reduced by 200 μm or more, the prognosis may be classified as “Strong response”, and if the decrease is below these values or if the thickness increases, the prognosis may be classified as “Weak response”. In another embodiment, in the extracting of the peripheral lesion information (S300), in the case of a patient treated with a steroid therapeutic agent, in the case of a female, the central retinal thickness after the first treatment is compared, and when the initial thickness is 305 to 379 μm, the central retinal thickness is reduced by 50 μm or more, when the initial thickness is 380 to 479 μm, the central retinal thickness is reduced by 100 μm or more, and when the initial thickness is 480 μm or more, the central retinal thickness is reduced by 200 μm or more, the prognosis may be classified as “Strong response”, and if the decrease is below these values or if the thickness increases, the prognosis may be classified as “Weak response”.

In the extracting of the peripheral lesion information (S300), five slices of two sheets on each side based on the slice of the center of the macula may be extracted from the OCT image, the center of the macula may be extracted as ROI using a retinal region mask from the extracted five slices, and the main lesion information may be extracted in the ROI using a main lesion mask.

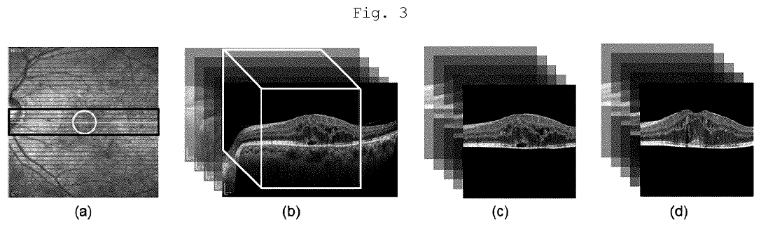

FIG. 3 illustrates an OCT image preprocessing process performed in the step S300 of extracting main lesion information according to an embodiment of the present invention. Referring to FIG. 3, in the extracting of the peripheral lesion information (S300), a total of five slices, two pieces on both sides based on the central slice of the macula, may be extracted from the OCT image in order to use the OCT slice in which swelling is photographed in the macula for learning in consideration of the photographing interval of the OCT image and the diameter of the macula.

Referring to FIG. 3A, a yellow circle indicates a macular region, and indicates five slice positions selected by a red box in an image. Referring to FIG. 3B, in the extracting of the peripheral lesion information (S300), a central region of the macula excluding the optic nerve bundle and the general retinal layer may be extracted, such as a red box, in order to extract ROI from each slice. Referring to FIG. 3C, in the extracting of the peripheral lesion information (S300), the vitreous body region and the choroid region may be removed, and this may be used as a final OCT image.

Referring to FIG. 3D, regions of Subretinal Fluid (SRF), Intraretinal Fluid (IRF), and Hyper-Reflective Dots (HRD), which are major lesions, may be masked on the OCT image. Various characteristic information such as area, perimeter, number, distribution degree, brightness, and contrast of the lesion were extracted by applying an image analysis technique to the lesion area.

In the step S500 of training the multi-modal deep learning model, the multi-modal deep learning model may be trained by inputting the OCT image, the clinical information, and the one or more main lesion features, and outputting the intravitreal injection treatment prognosis of the diabetic macular edema patient.

The multi-modal deep learning model may be a structure in which a Convolutional Neural Network (CNN) for extracting features of an OCT image and a Deep Neural Network (DNN) for extracting features of clinical information and major lesion information are combined.

In the step S500 of training the multi-modal deep learning model, in order to select an optimal feature, the importance of each feature may be evaluated by using the SHAP (SHapley Additive explanations) value calculated in the single modality training process. In the step of training the multi-modal deep learning model (S500), a change in performance for 11 combinations may be checked by excluding features having low importance in units of 5, and an optimal feature combination capable of maximizing the accuracy and efficiency of the model may be selected. Through this, in the step S500 of training the multi-modal deep learning model, it is possible to identify main features that affect prediction performance.

As the convolutional neural network for feature extraction of the OCT image, DenseNet121, MobileNet-V3, WideResNet50, and EfficientNet-B2, which are CNN-based models, may be used. In the deep neural network for extracting features of clinical information and major lesion information (table data), various hyper-parameter combinations are experimented through a grid search method, so that the number of layers, hidden units, batch size, training rate, and the like may be adjusted, and accordingly, a model most suitable for the characteristics of table data may be applied. In the present invention, the model was constructed by adopting the feature fusion method, the characteristics independently extracted from each modality were integrated, and the final classification was performed through the classifier layer.

In the step S500 of training the multi-modal deep learning model, training may be performed in an k-fold cross validation method to enhance the generalization ability of the model and prevent overfitting. The training process was largely divided into two stages. First, a single modality learning was performed on each data to obtain a characteristic extraction weight suitable for data characteristics. The CNN-based model was used for OCT images, and fine tuning was performed in a pre-trained state with the ImageNet dataset. Training was performed by applying data augmentation techniques to simulate various types of OCT images. In the case of table data, the DNN structure was directly constructed, and the optimal combination for the number of layer, hidden unit, learning-rate, and batch size was searched and trained through the grid-search method. As the next step, the optimized weight learned prior to the characteristic extraction layer of each data is loaded into the multi-modal model as it is and frozen. After that, only the classifier layer was studied.

In the step S500 of training the multi-modal deep learning model, the trained model may be evaluated. The model was evaluated on a patient-by-patient basis. In the step of training the multi-modal deep learning model (S500), each prediction probability is obtained for each of the five slices for each patient, and a representative prediction value of the corresponding patient may be determined with the maximum probability among them and compared with the label. In the step of training the multi-modal deep learning model (S500), since the type or size of the lesion appearing for each slice is different, even in at least one of the five slices, if a major symptom is detected, it may be reflected in prediction.

In the step S700 of predicting the intravitreal injection treatment prognosis, the intravitreal injection treatment prognosis may be predicted by inputting the OCT image, clinical information, and major lesion characteristics of a specific diabetic macular edema patient to the trained multi-modal deep learning model.

In the predicting of the intravitreal injection treatment prognosis (S700), a first feature may be extracted by inputting an OCT image to a convolutional neural network, a second feature may be extracted by inputting the clinical information and the main lesion information to a deep neural network, and the first feature and the second feature may be combined in a late-fusion manner and then input to a fully-connected network (FCN) to predict the intravitreal injection treatment prognosis of a diabetic macular edema patient.

In the step S700 of predicting the prognosis of intravitreal injection treatment, the prognosis may be predicted based on the amount of change in the central retinal thickness after one or more intravitreal injection treatments. In an embodiment, in the predicting of the prognosis of intravitreal injection treatment (S700), in the case of a patient treated with an anti-VEGF therapeutic agent, the prognosis may be predicted as “Good response” if the central retinal thickness immediately after treatment is reduced by 50 μm or more, and “Bad response” if the central retinal thickness is less than or increased. In another embodiment, in the predicting of the prognosis of intravitreal injection treatment (S700), in the case of a patient treated with a steroid therapeutic agent, in the case of a male, the thickness of the central retina after the first treatment is compared, and when the initial thickness is 320 to 374 μm, the thickness is reduced by 50 μm or more, when the initial thickness is 375 to 494 μm, the thickness is reduced by 100 μm or more, and when the thickness is 495 μm μm or more, the thickness is reduced by 200 μm or more, the prognosis may be predicted as “Strong response”, and when the thickness is less or increased, the prognosis may be predicted as “Weak response”. In another embodiment, in the step of predicting the prognosis of intravitreal injection treatment (S700), in the case of a patient treated with a steroid therapeutic agent, in the case of a female, the thickness of the central retina after the first treatment is compared, and when the initial thickness is 305 to 379 μm, the thickness is reduced by 50 μm or more, when the initial thickness is 380 to 479 μm, the thickness is reduced by 100 μm or more, and when the thickness is 480 μm μm or more, the thickness is reduced by 200 μm or more, the prognosis may be predicted as “Strong response”, and when the thickness is less or increased, the prognosis may be predicted as “Weak response”.

### Simulation Results: First Embodiment (Anti-VEGF Injection Treatment)

The dataset used in this simulation consists of data collected from 107 patients who were treated for diabetic macular edema. For the statistical and clinical characteristics of the dataset, treatment responsiveness was divided into groups of “Good response” and “Bad response” based on the change in retinal center thickness, as shown in Table 1 below. After three treatments, 69 patients with good progress and 38 patients with no change or worsening were classified. The average age of patients is in their early 60s, and men are older than women. The group with poor response was generally at a higher stage of diabetic retinopathy. There were more patients with natural lens than with artificial lens, and more patients had no history of PRP surgery or injection treatment.

TABLE 1
Demographics of clinical dataset

| | Good response | Bad response |
|---|---|---|
| | (n = 69) | (n = 38) |
| Age (year) | 61.26 ± 11.14 | 61.63 ± 9.12 |
| Sex | | |
| Male | 41 | 24 |
| Female | 28 | 14 |
| Presence of Hypertension | | |
| Negative | 41 | 20 |
| Positive | 28 | 18 |
| DR stage | | |
| mild NPDR | 5 | 0 |
| moderate NPDR | 9 | 3 |
| severe NPDR | 33 | 21 |
| PDR | 22 | 14 |
| Lens status | | |
| phakia | 49 | 25 |
| pseudophakia | 20 | 13 |
| PRP history | | |
| Negative | 55 | 30 |
| Positive | 14 | 8 |
| Previous injections history | | |
| Negative | 52 | 28 |
| Positive | 17 | 10 |
| Central Retinal Thickness (μm) | 455.12 ± 92.21 | 397.84 ± 71.23 |
| Intraocular Pressure (mmHg) | 17.00 ± 3.77 | 16.55 ± 2.90 |

PRP = Pan-Retinal Photocoagulation,
DR stage = Diabetic Retinopathy Stages,
NPDR = Non-Proliferative Diabetic Retinopathy,
PDR = Proliferative Diabetic Retinopathy

For the OCT image, 5 slices were extracted from 107 patients through the preprocessing process, and a total of 535 images were used for learning. As for the lesion area characteristic information extracted from the OCT image, 15 types of characteristics related to the area, perimeter, distribution, pixel intensity, and image texture for each lesion type, and 46 types of characteristics per slice were extracted by adding the area of the entire retinal area. In addition, nine types of clinical information were added, and table data were constructed with a total of 55 characteristics and used for learning.

Since the learning was conducted in the 5-fold cv method, the learning set and the evaluation set were divided into an 8:2 ratio, consideration was given to not creating an unbalance of the label, and care was taken not to include the learning set and the evaluation set in the image of one patient.

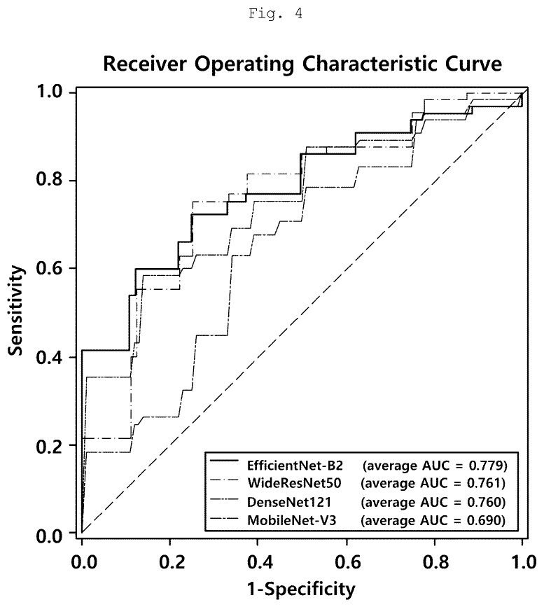

FIG. 4 is a performance index of an CNN-based model according to a first embodiment of the present invention. Referring to FIG. 4, four CNN-based models (DenseNet121, MobileNet-V3, WideResNet50, and EfficientNet-B2) were trained and evaluated with OCT images. The AUROC was compared by drawing the average ROC curve for 5-fold CV as shown in FIG. 4, and other performance indicators are summarized in Table 2 below.

TABLE 2
Performance comparison of models trained by OCT image data

| | | | | | F1- |
|---|---|---|---|---|---|
| Model | Accuracy | Sensitivity | Specificity | Precision | score |
| DenseNet121 | 0.747 | 0.862 | 0.567 | 0.761 | 0.802 |
| MobileNet-V3 | 0.663 | 0.785 | 0.467 | 0.709 | 0.740 |
| WideResNet50 | 0.738 | 0.830 | 0.592 | 0.760 | 0.792 |
| EfficientNet-B2 | 0.785 | 0.656 | 0.796 | 0.785 | 0.781 |

DenseNet121 showed an average AUROC of 0.760, and the sensitivity was the highest at 0.862. F1 score was the highest at 0.802. Accuracy (0.747) and precision (0.761) were the second highest. MobileNet-V3 had the lowest AUROC (0.690) and accuracy (0.663), specificity (0.467), precision (0.709) and F1 score (0.740), and sensitivity (0.785) recorded the third. Overall, lower performance was confirmed compared to other models. WideResNet50 showed the second highest AUROC at 0.761, but showed lower performance than DenseNet121 in the rest of the evaluation index except for specificity (0.592). EfficientNet-B2 showed the highest performance with AUROC 0.779, and accuracy showed the highest performance with 0.785. The specificity was 0.796, which was much higher than other models, and the precision was also the best. On the other hand, sensitivity (0.656) was the lowest. When comparing the performance of the four models, EfficientNet-B2 showed the best performance, and it was selected as an backbone model that extracts the characteristics of OCT images from multi-modal learning.

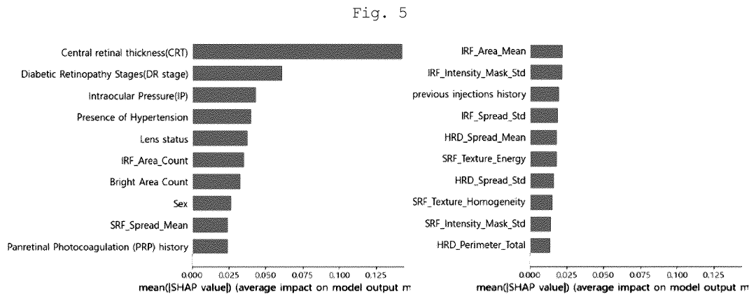

FIG. 5 shows the feature importance of the top 20 combinations according to the first embodiment of the present invention. Referring to FIG. 5, how much clinical information and lesion characteristic information of 55 species affect learning of the DNN model was confirmed through SHAP, and a combination for performance improvement was searched. The top 5 of importance were clinical information such as CRT, DR stage, IP, presence of hypertension, and lens condition. From the 6th place, past history and lesion characteristics were shown. With 4 types of IRF, 3 types of SRF, and 4 types of HRD, it can be seen that lesion characteristics play an important role in learning the model. Performance for each feature combination according to feature importance is shown in Table 3 below.

TABLE 3
TABLE 3. Performance of Single-Modal Models
Based on the Number of Table Data Features

| | | Accu- | Sensi- | Speci- | Preci- | F1- |
|---|---|---|---|---|---|---|
| Data | AUROC | racy | tivity | ficity | sion | score |
| 55 features | 0.717 | 0.632 | 0.662 | 0.586 | 0.643 | 0.631 |
| 50 features | 0.734 | 0.684 | 0.714 | 0.638 | 0.695 | 0.684 |
| 45 features | 0.748 | 0.671 | 0.680 | 0.657 | 0.688 | 0.671 |
| 40 features | 0.719 | 0.636 | 0.646 | 0.619 | 0.653 | 0.636 |
| 35 features | 0.725 | 0.682 | 0.695 | 0.662 | 0.696 | 0.683 |
| 30 features | 0.727 | 0.649 | 0.702 | 0.567 | 0.655 | 0.646 |
| 25 features | 0.768 | 0.690 | 0.720 | 0.643 | 0.702 | 0.688 |
| 20 features | 0.779 | 0.723 | 0.800 | 0.605 | 0.724 | 0.719 |
| 15 features | 0.768 | 0.705 | 0.735 | 0.657 | 0.711 | 0.704 |
| 10 features | 0.693 | 0.607 | 0.622 | 0.586 | 0.622 | 0.610 |
| 5 features | 0.739 | 0.729 | 0.800 | 0.619 | 0.738 | 0.724 |

The model with 20 clinical characteristics achieved the highest average AUROC (0.779) and sensitivity (0.800), while accuracy (0.723), precision (0.724), and F1 score (0.719) were the second. When learning with a combination of five, accuracy (0.729), precision (0.738), and F1 score (0.724) were the highest. Learning with 10 combinations showed the lowest overall performance, and learning with 55 combinations showed the second lowest performance. It was confirmed that the performance of the model is generally good when the number of characteristics is less than when the number of characteristics is large. In all combinations, sensitivity was higher than specificity.

In order to confirm the performance of the optimal multi-modal model, we learned the combination according to the characteristic importance of the table data and the OCT image data together. To improve the model's predictive performance for treatment response classification, multi-modal learning was conducted and evaluated using table data and OCT data. For OCT images, the efficientnet-b2 selected from the single modality training was used for feature extraction, and the feature combinations of table data were trained and evaluated for 11 combinations by removing the lower 5 each, the same as when the single modality training was performed. The learning results are shown in Table 4 below.

TABLE 4
TABLE 4. Performance of Multi-Modal Models
Based on the Number of Table Data Features

| Data | AUROC | Accuracy | Sensitivity | Specificity | Precision | F1-score |
|---|---|---|---|---|---|---|
| 55 features + OCT | 0.749 | 0.700 | 0.708 | 0.692 | 0.785 | 0.735 |
| 50 features + OCT | 0.885 | 0.859 | 0.800 | 0.953 | 0.967 | 0.862 |
| 45 features + OCT | 0.960 | 0.926 | 0.938 | 0.908 | 0.939 | 0.938 |
| 40 features + OCT | 0.962 | 0.953 | 0.969 | 0.928 | 0.956 | 0.962 |
| 35 features + OCT | 0.944 | 0.906 | 0.862 | 0.975 | 0.980 | 0.915 |
| 30 features + OCT | 0.929 | 0.907 | 0.892 | 0.931 | 0.950 | 0.919 |
| 25 features + OCT | 0.933 | 0.926 | 0.892 | 0.975 | 0.982 | 0.933 |
| 20 features + OCT | 0.956 | 0.898 | 0.892 | 0.906 | 0.934 | 0.910 |
| 15 features + OCT | 0.921 | 0.897 | 0.892 | 0.908 | 0.938 | 0.912 |
| 10 features + OCT | 0.945 | 0.888 | 0.892 | 0.881 | 0.922 | 0.906 |
| 5 features + OCT | 0.927 | 0.868 | 0.892 | 0.833 | 0.893 | 0.889 |

Looking at the performance indicators, by learning the two data together, the model performance was significantly improved compared to when each data was learned alone. Excluding the case of learning with 55 characteristics, the remaining combination showed an improvement of AUROC 0.15˜0.25. The model learned with 40 characteristics and OCT data showed the highest performance, with the highest AUROC (0.962), accuracy (0.953), sensitivity (0.969) and F1 score (0.962). Specificity was the highest at 0.975 in the case of using 35 and 25 characteristics. Precision was the highest at 0.982 for each of the 25 characteristics and the case of learning with OCT. When 5 to 30 characteristics were used, the sensitivity was all 0.892, showing no change. Although the performance of each fold varied according to the change in the number of characteristics, it did not significantly affect the average performance. Overall, there was a significant performance improvement in multi-modal learning, and in particular, specificity improved significantly by an average of 0.28 compared to single modality learning performance.

### Simulation Results: Second Example (Steroid Injection Treatment)

The data net used in this simulation consists of data collected from 45 patients undergoing diabetic macular edema treatment. For the statistical and clinical characteristics of the dataset, treatment responsiveness was divided into groups of “Strong response” and “Weak response” based on the change in retinal center thickness, as shown in Table 5 below. After one treatment, 37 patients with strong response to the therapeutic agent, and 8 patients with no response or weak response were classified.

TABLE 5
Demographics of clinical dataset

| | Strong response | Week response |
|---|---|---|
| | (n = 37) | (n = 8) |
| Age (year) | 58.89 ± 8.97 | 58.68 ± 9.00 |
| Sex | | |
| Male | 18 | 1 |
| Female | 19 | 7 |
| Presence of Hypertension | | |
| Negative | 18 | 3 |
| Positive | 19 | 5 |
| DR stage | | |
| mild NPDR | 0 | 0 |
| moderate NPDR | 5 | 0 |
| severe NPDR | 12 | 4 |
| PDR | 20 | 4 |
| Lens status | | |
| phakia | 18 | 2 |
| pseudophakia | 19 | 6 |
| PRP history | | |
| Negative | 22 | 6 |
| Positive | 15 | 2 |
| Previous injections history | | |
| Negative | 1 | 0 |
| Positive | 36 | 8 |
| Central Retinal Thickness (μm) | 501.00 ± 116.99 | 497.36 ± 116.47 |
| Intraocular Pressure (mmHg) | 15.55 ± 3.33 | 15.59 ± 3.35 |

PRP = Pan-Retinal Photocoagulation,
DR stage = Diabetic Retinopathy Stages,
NPDR = Non-Proliferative Diabetic Retinopathy,
PDR = Proliferative Diabetic Retinopathy

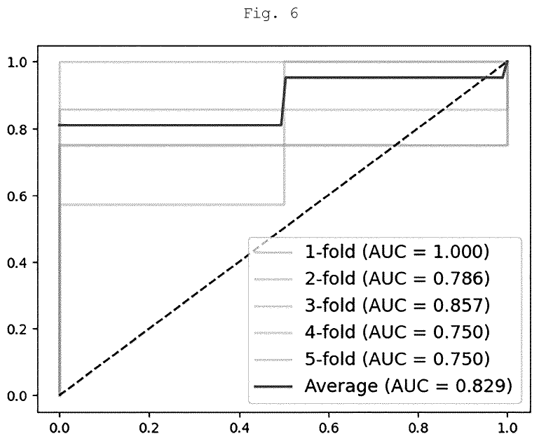

FIG. 6 shows a performance index of an CNN-based model according to a second embodiment of the present invention. Referring to FIG. 6, the EfficientNet-B2 model having the best effect in the previous simulation was trained and evaluated. The AUROC was compared by drawing the average ROC curve for 5-fold CV as shown in FIG. 6, and other performance indicators are summarized in Table 6 below.

TABLE 6
Performance comparison of models trained by OCT image data

| | | | | | F1- |
|---|---|---|---|---|---|
| Model | Accuracy | Sensitivity | Specificity | Precision | score |
| EfficientNet-B2 | 0.829 | 0.896 | 0.400 | 0.877 | 0.880 |

EfficientNet-B2 was evaluated with Accuracy of 0.829, Sensitivity of 0.896, Specificity of 0.400, Precision of 0.877, and F1-score of 0.880. It is evaluated to be superior to the results of Simulation 1. The other Specificity was recorded lower than the other indicators, which seems to be the result of the relatively small amount of data in “Weak response”.

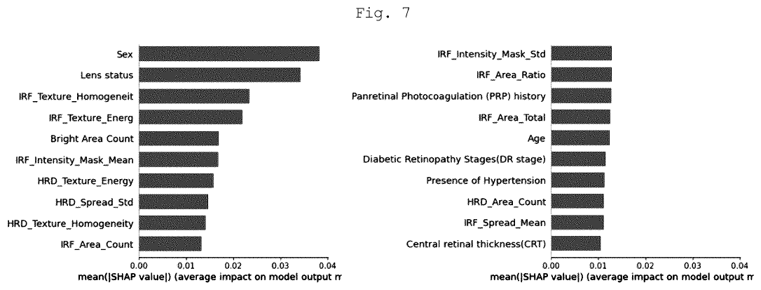

FIG. 7 shows the feature importance of the top 20 combinations according to the second embodiment of the present invention. Referring to FIG. 7, how much the 55 types of clinical information and lesion characteristic information affect learning of the DNN model was confirmed through SHAP, and a combination for performance improvement was searched. The top 5 in importance were clinical information such as SEX, Lens status, IRF_Texture_Homogeneit, IRF_Texture_Energ, and Bright Area Count. Performance for each feature combination according to feature importance is shown in Table 7 below.

TABLE 7
TABLE 7. Performance of Single-Modal Models
Based on the Number of Table Data Features

| | | Accu- | Sensi- | Speci- | Preci- | F1- |
|---|---|---|---|---|---|---|
| Data | AUROC | racy | tivity | ficity | sion | score |
| 55 features | 0.778 | 0.858 | 0.956 | 0.400 | 0.838 | 0.835 |
| 50 features | 0.795 | 0.849 | 0.956 | 0.340 | 0.814 | 0.820 |
| 45 features | 0.791 | 0.840 | 0.935 | 0.400 | 0.803 | 0.817 |
| 40 features | 0.692 | 0.840 | 0.946 | 0.320 | 0.797 | 0.810 |
| 35 features | 0.762 | 0.822 | 0.925 | 0.320 | 0.818 | 0.809 |
| 30 features | 0.739 | 0.818 | 0.941 | 0.240 | 0.751 | 0.781 |
| 25 features | 0.710 | 0.840 | 0.944 | 0.320 | 0.784 | 0.806 |
| 20 features | 0.669 | 0.818 | 0.927 | 0.280 | 0.775 | 0.793 |
| 15 features | 0.742 | 0.849 | 0.950 | 0.360 | 0.838 | 0.826 |
| 10 features | 0.746 | 0.858 | 0.946 | 0.420 | 0.852 | 0.843 |
| 5 features | 0.767 | 0.822 | 0.919 | 0.340 | 0.850 | 0.798 |

The model with 50 clinical characteristics achieved the highest average AUROC (0.795) and sensitivity (0.956), and accuracy (0.849) recorded the second. When learning with 10 combinations, accuracy (0.858), precision (0.852), and F1 score (0.843) were the highest. Learning with 20 combinations showed the lowest overall performance, and learning with 30 combinations showed the second lowest performance.

In order to confirm the performance of the optimal multi-modal model, we learned the combination according to the characteristic importance of the table data and the OCT image data together. To improve the model's predictive performance for treatment response classification, multi-modal learning was conducted and evaluated using table data and OCT data. For OCT images, the efficientnet-b2 selected from the single modality training was used for feature extraction, and the feature combinations of table data were trained and evaluated for 11 combinations by removing the lower 5 each, the same as when the single modality training was performed. The learning results are shown in Table 8 below.

TABLE 8
TABLE 8. Performance of Multi-Modal Models
Based on the Number of Table Data Features

| Data | AUROC | Accuracy | Sensitivity | Specificity | Precision | F1-score |
|---|---|---|---|---|---|---|
| 55 features + OCT | 0.791 | 0.778 | 0.896 | 0.200 | 0.848 | 0.868 |
| 50 features + OCT | 0.873 | 0.889 | 0.971 | 0.400 | 0.902 | 0.935 |
| 45 features + OCT | 0.752 | 0.778 | 0.896 | 0.200 | 0.848 | 0.868 |
| 40 features + OCT | 0.764 | 0.778 | 0.896 | 0.200 | 0.848 | 0.868 |
| 35 features + OCT | 0.804 | 0.822 | 0.921 | 0.300 | 0.877 | 0.896 |
| 30 features + OCT | 0.793 | 0.778 | 0.896 | 0.200 | 0.848 | 0.868 |
| 25 features + OCT | 0.764 | 0.800 | 0.918 | 0.200 | 0.849 | 0.881 |
| 20 features + OCT | 0.754 | 0.778 | 0.896 | 0.200 | 0.848 | 0.868 |
| 15 features + OCT | 0.780 | 0.844 | 0.946 | 0.300 | 0.880 | 0.910 |
| 10 features + OCT | 0.884 | 0.822 | 0.946 | 0.200 | 0.855 | 0.896 |
| 5 features + OCT | 0.818 | 0.800 | 0.896 | 0.300 | 0.873 | 0.881 |

Looking at the performance indicators, it can be seen that the overall performance of the model has improved compared to when each data is learned alone by learning the two data together.

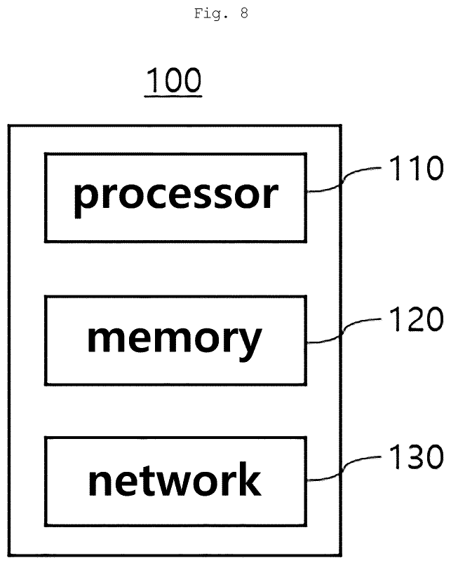

FIG. 8 is a configuration diagram of a device 100 for predicting a prognosis of intravitreal injection therapy according to an embodiment of the present invention. Referring to FIG. 8, the configuration of the device 100 for predicting the prognosis of the illustrated intravitreal injection treatment is only a simplified example. In an embodiment of the present invention, the device 100 for predicting the prognosis of intravitreal injection treatment may include other configurations for performing the computing environment of the device 100, and only some of the disclosed configurations may configure the device 100.

The device 100 for predicting the prognosis of intravitreal injection treatment may include a processor 110 including one or more cores, a memory 120, and a network 130.

The processor 110 may include one or more cores, and may include a processor for data analysis and deep learning of a central processing unit (CPU), a general purpose graphics processing unit (GPGPU), a tensor processing unit (TPU), and the like of a computing device. The processor 110 may read a computer program stored in the memory 120 to perform data processing for machine learning according to an embodiment of the present disclosure. According to an embodiment of the present disclosure, the processor 110 may perform an operation for learning a neural network. The processor 110 may perform calculation for training of a neural network, such as processing of input data for training in DL (deep learning), feature extraction from the input data, error calculation, and weight update of the neural network using backpropagation. At least one of CPU, GPGPU, and TPU of the processor 110 may process learning of a network function. For example, CPU and GPGPU may process learning of a network function and data classification using the network function together. In addition, in an embodiment of the disclosure, learning of a network function and data classification using the network function may be processed by using processors of a plurality of computing devices together. In addition, the computer program performed in the computing device according to an embodiment of the present disclosure may be a CPU, GPGPU, or TPU executable program.

The processor 110 may receive OCT images and clinical information of a plurality of diabetic macular edema patients. The processor 110 may perform the step S100 of receiving the above-described OCT image and clinical information.

The processor 110 may extract one or more pieces of main lesion information from the OCT image. The processor 110 may perform the step S300 of extracting the above-described peripheral lesion information.

The processor 110 may train a multi-modal deep learning model by inputting the OCT image, the clinical information, and the one or more main lesion features as inputs, and outputting an intravitreal injection treatment prognosis of a diabetic macular edema patient. The processor 110 may perform step S500 of training the above-described multi-modal deep learning model.

The processor 110 may predict the prognosis of intravitreal injection treatment by inputting the OCT image, clinical information, and major lesion characteristics of a specific diabetic macular edema patient to the trained multi-modal deep learning model. The processor 110 may perform the step S700 of predicting the above-described intravitreal injection treatment prognosis.

The memory 120 may store any type of information generated or determined by the processor 110 and any type of information received by the network 130.

The memory 120 may include at least one storage medium of a flash memory type, a hard disk type, a multimedia card micro type, a card type memory (e.g., SD or XD memory), a Random Access Memory (RAM), a Static Random Access Memory (SRAM), a Read-Only Memory (ROM), an Electrically Erasable Programmable Read-Only Memory (EEPROM), a Programmable Read-Only Memory (PROM), a magnetic memory, a magnetic disk, and an optical disk. The computing device 100 may operate in relation to a web storage that performs a storage function of the memory 120 on the Internet. The description of the memory is only an example, and the present disclosure is not limited thereto.

The network 130 may use any type of known wired/wireless communication system. The network 130 may receive an OCT image or the like from a related device or a related system.

The network 130 may transmit and receive information processed by the processor 110, a user interface, and the like through communication with another terminal. For example, the network 130 may provide the user interface generated by the processor 100 to the client (e.g a user terminal). In addition, the network 130 may receive an external input of the user applied to the client and transmit the external input to the processor 110. In this case, the processor 110 may process operations such as outputting, modifying, changing, adding, etc. of information provided through the user interface based on the user's external input received from the network 130.

Meanwhile, the device 100 for predicting the prognosis of intravitreal injection treatment according to an embodiment of the disclosure may include a server as a computing system for transmitting and receiving information through communication with a client. In this case, the client may be any type of terminal capable of accessing the server.

In an additional embodiment, the device 100 for predicting the prognosis of intravitreal injection treatment may include any type of terminal that receives data resources generated by any server and performs additional information processing.

A computer program for predicting a prognosis of intravitreal injection treatment, which is another embodiment of the present invention, may include an operation of receiving a OCT image and clinical information, an operation of extracting peripheral lesion information, an operation of training a multi-modal deep learning model, and an operation of predicting an intravitreal injection treatment prognosis. The computer program for predicting the prognosis of intravitreal injection treatment may include instructions stored in a computer-readable storage medium to cause the computer to perform the following operations.

The operation of receiving the OCT image and clinical information may include receiving the OCT image and clinical information of a plurality of diabetic macular edema patients. The operation of receiving the OCT image and the clinical information refers to an operation performed in the step S100 of receiving the OCT image and the clinical information.

The operation of extracting the peripheral lesion information may extract one or more pieces of main lesion information from the OCT image. The operation of extracting the peripheral lesion information refers to an operation performed in the step S300 of extracting the peripheral lesion information.

The operation of training the multi-modal deep learning model may include training the multi-modal deep learning model by inputting the OCT image, the clinical information, and the one or more main lesion features as inputs, and outputting a prognosis of intravitreal injection treatment of a diabetic macular edema patient as an output. The operation of training the multi-modal deep learning model refers to an operation performed in the step S500 of training the multi-modal deep learning model described above.

The operation of predicting the intravitreal injection treatment prognosis may predict the intravitreal injection treatment prognosis by inputting the OCT image, clinical information, and major lesion characteristics of a specific diabetic macular edema patient to the trained multi-modal deep learning model. The operation of predicting the intravitreal injection treatment prognosis refers to an operation performed in the step S700 of predicting the intravitreal injection treatment prognosis described above.

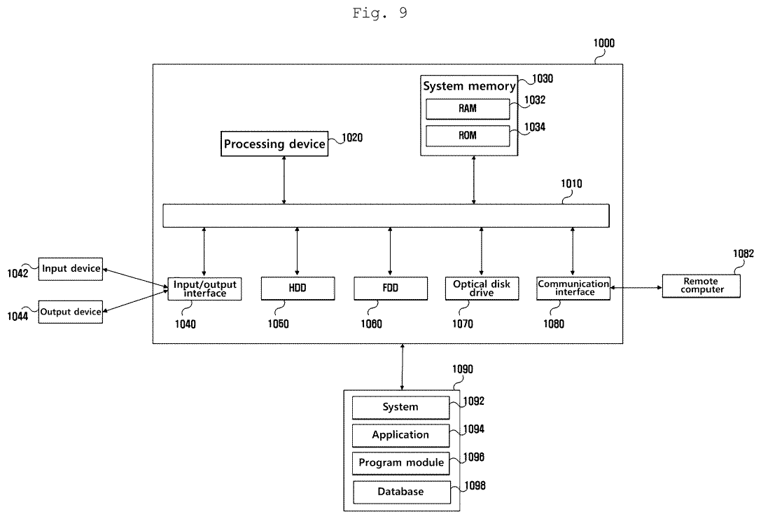

FIG. 9 is a schematic diagram of a computing environment according to an embodiment of the present invention.

While the present disclosure has been described above as being generally implemented by a computing device, those skilled in the art will appreciate that the present disclosure may be implemented as a combination of hardware and software and/or in combination with computer-executable instructions and/or other program modules that may be executed on one or more computers.

In general, program modules include routines, programs, components, data structures, and the like that perform particular tasks or implement particular abstract data types. It will also be appreciated by those skilled in the art that the methods of the present disclosure may be practiced in other computer system configurations, including single-processor or multiprocessor computer systems, minicomputers, mainframe computers, as well as personal computers, handheld computing devices, microprocessor-based or programmable home appliances, and the like, each of which may operate in connection with one or more associated devices.

The described embodiments of the present disclosure may also be practiced in a distributed computing environment where certain tasks are performed by remote processing devices connected via a communication network. In a distributed computing environment, program modules may be located in both local and remote memory storage devices.

A computer typically includes a variety of computer-readable media. Any computer-accessible medium may be a computer-readable medium, including volatile and non-volatile media, transitory and non-transitory media, and removable and non-removable media. By way of example and not limitation, computer-readable media may include computer-readable storage media and computer-readable transmission media. Computer-readable storage media includes volatile and non-volatile media, transitory and non-transitory media, removable and non-removable media, implemented in any method or technology for storing information such as computer-readable instructions, data structures, program modules, or other data. Computer-readable storage media includes, but is not limited to, RAM, ROM, EEPROM, flash memory or other memory technology, CD-ROM, digital video disk (DVD) or other optical disk storage, magnetic cassettes, magnetic tapes, magnetic disk storage or other magnetic storage devices, or any other medium that can be accessed by a computer and used to store desired information.

Computer-readable transmission media typically embody computer-readable instructions, data structures, program modules or other data in a modulated data signal, such as a carrier wave or other transport mechanism, and include all information delivery media. The term “demodulated data signal” refers to a signal in which one or more of the characteristics of the signal are set or changed to encode information in the signal. By way of example and not limitation, computer-readable transmission media include wired media such as wired networks or direct-wired connections, and wireless media such as acoustic, RF, infrared, and other wireless media. It is assumed that a combination of any of the above-described media is also included within the scope of the computer-readable transmission medium.

An exemplary environment is shown that implements various aspects of the present disclosure, including a computer 1000, the computer 1000 including a processing device 1020, a system memory 1030, and a system bus 1010. The system bus 1010 couples system components including, but not limited to, the system memory 1030 to the processing device 1020. The processing device 1020 may be any of various commercial processors. Dual processor and other multi-processor architectures may also be used as the processing device 1020.

The system bus 1010 may be any of several types of bus structures that may additionally be interconnected to a memory bus, a peripheral bus, and a local bus using any of a variety of commercial bus architectures. The system memory 1030 includes read only memory (ROM) 1034 and random access memory (RAM) 1032. A basic input/output system (BIOS) is stored in a non-volatile memory 1034 such as ROM, EPROM, EEPROM BIOS, etc., which includes a basic routine to help transfer information between components in the computer 1000 when starting up. The RAM 1032 may also include a high-speed RAM, such as a static RAM, for caching data.

The computer 1000 also includes an embedded hard disk drive (HDD) 1050 (e.g., EIDE, SATA), wherein the embedded hard disk drive 1050 may also be configured for external use within a suitable chassis (not shown), a magnetic floppy disk drive (FDD) 1060 (e.g., for reading from or writing to a removable diskette), and an optical disk drive 1070 (e.g., for reading a CD-ROM disk or reading from or writing to other high capacity optical media such as DVD). The hard disk drive 1050, the magnetic disk drive 1060, and the optical disk drive 1070 may be connected to the system bus 1010 by a hard disk drive interface, a magnetic disk drive interface, and an optical drive interface, respectively. The interface for implementing an external drive includes at least one or both of a Universal Serial Bus (USB) and an IEEE 1394 interface technology.

These drives and associated computer-readable media provide non-volatile storage of data, data structures, computer-executable instructions, and the like. For computer 1000, drives and media correspond to storing any data in a suitable digital format. While the description of the computer-readable medium above refers to a HDD, a removable magnetic disk, and a removable optical medium such as a CD or DVD, those skilled in the art will appreciate that other tangible media readable by a computer, such as zip drives, magnetic cassettes, flash memory cards, cartridges, and the like, may also be used in an exemplary operating environment, and any such medium may include computer-executable instructions for performing the methods of the present disclosure.

A number of program modules may be stored in the drive and RAM 1032, including an operating system 1092, one or more application programs 1094, other program modules 1096, and a database 1098. All or a portion of the operating system, applications, modules, and/or data may also be cached in the RAM 1032. It will be appreciated that the present disclosure may be implemented in various commercially available operating systems or combinations of operating systems.

A user may enter commands and information into the computer 1000 through one or more wired/wireless input devices 1042, such as a keyboard and a pointing device such as a mouse. Other input devices (not shown) may include a microphone, a IR remote control, a joystick, a game pad, a stylus pen, a touch screen, and the like. These and other input devices are often connected to the processing device 1020 via an input/output interface 1040 that is connected to the system bus 1010, but may be connected by other interfaces such as a parallel port, an IEEE 1394 serial port, a game port, a USB port, an IR interface, and the like.

A monitor or other type of display device is also connected to the system bus 1010 through an interface such as a video adapter. In addition to monitors, computers generally include other peripheral output devices (not shown) such as speakers, printers, and the like.

The computer 1000 may operate in a networked environment using logical connections to one or more remote computers, such as remote computer(s) 1082 via wired and/or wireless communications. The remote computer(s) 1082 may be a workstation, a computing device computer, a router, a personal computer, a portable computer, a microprocessor-based entertainment device, a peer device, or other conventional network node, and may generally include many or all of the components described for the computer 1000. The logical connection includes a local area network (LAN) and/or a wired/wireless connection to a larger network, for example, a wide area network (WAN). Such LAN and WAN networking environments are common in offices and businesses, and facilitate enterprise-wide computer networks, such as intranets, all of which may be connected to a global computer network, e.g., the Internet.

When used in a LAN networking environment, the computer 1000 is connected to a local network (not shown) via a wired and/or wireless communication network interface or adapter (not shown). An adapter (not shown) may facilitate wired or wireless communication to a LAN (not shown), the LAN (not shown) also including a wireless access point installed therein to communicate with the wireless adapter (not shown). When used in a WAN networking environment, the computer 1000 may include a modem (not shown), or has other means of establishing communication over a WAN (not shown), such as being connected to a communication computing device on a WAN (not shown), or via the Internet. A modem (not shown), which may be internal or external and a wired or wireless device, is connected to the system bus 1010 via a serial port interface (not shown). In a networked environment, program modules or portions thereof described for computer 1000 may be stored in a remote memory/storage device (not shown). It will be appreciated that the illustrated network connection is exemplary and other means of establishing communication links between computers may be used.

The computer 1000 is operable to communicate with any wireless device or object, e.g., a printer, scanner, desktop and/or portable computer, portable data assistant (PDA), communication satellite, any equipment or place associated with a wireless detectable tag, and telephone, which are arranged and operated in wireless communication. This includes at least Wi-Fi and Bluetooth wireless technology. Accordingly, the communication may be a predefined structure as in a conventional network or simply an ad hoc communication between at least two devices.

Wi-Fi (Wireless Fidelity) enables connection to the Internet, etc. without wired. The Wi-Fi is a wireless technology such as a cell phone that allows such devices, e.g., computers, to transmit and receive data indoors and outdoors, i.e. anywhere within the base station's call zone. The Wi-Fi network uses a wireless technology called IEEE 802.11 (a, b, g, etc.) to provide a secure, reliable, and high-speed wireless connection. Wi-Fi may be used to connect computers to each other, to the Internet, and to a wired network (using IEEE 802.3 or Ethernet). The Wi-Fi network may operate in unlicensed 2.4 and 5 GHz radio bands, e.g., at 11 Mbps (802.11a) or 54 Mbps (802.11b) data rates, or in products that include both bands (dual bands).

One of ordinary skill in the art of the present disclosure will understand that information and signals may be represented using any of a variety of different techniques and techniques. For example, data, instructions, instructions, information, signals, bits, symbols, and chips that may be referenced in the description above may be represented by voltages, currents, electromagnetic waves, magnetic fields or particles, optical fields or particles, or any combination thereof.

One of ordinary skill in the art of the present disclosure will understand that the various illustrative logical blocks, modules, processors, means, circuits, and model steps described in connection with the embodiments disclosed herein can be implemented by electronic hardware, various forms of program or design code (referred to herein for convenience as software), or a combination of both. To clearly illustrate this interoperability of hardware and software, various illustrative components, blocks, modules, circuits, and steps have been described above generally in relation to their functionality. Whether these features are implemented as hardware or software depends on the design constraints imposed on a particular application and the entire system. Those skilled in the art of the present disclosure may implement the functions described in various ways for each specific application, but such implementation decisions should not be construed as being outside the scope of the present disclosure.

The various embodiments presented herein may be implemented as a method, device, or article of manufacture using standard programming and/or engineering techniques. The term article of manufacture includes a computer program, carrier, or media accessible from any computer-readable storage device. For example, computer-readable storage media includes, but is not limited to, magnetic storage devices (e.g., hard disk, floppy disk, magnetic strip, etc.), optical disks (e.g., CD, DVD, etc.), smart cards, and flash memory devices (e.g., EEPROM, cards, sticks, key drives, etc.). In addition, the various storage media presented herein include one or more devices and/or other machine-readable media for storing information.

It is to be understood that the specific order or hierarchy of steps in the presented processes is an example of exemplary approaches. Based on the design priorities, it is understood that a particular order or hierarchy of steps in processes may be rearranged within the scope of the present disclosure. The appended method claims provide elements of various steps in sample order but do not imply that they are limited to the particular order or hierarchy presented.

The description of the presented embodiments is provided to be able to use or implement the present disclosure by those of ordinary skill in the art of any present disclosure. Various variations of these embodiments will be apparent to those of ordinary skill in the art of the disclosure, and the general principles defined herein may be applied to other embodiments without departing from the scope of the disclosure. Thus, the present disclosure is not limited to the embodiments set forth herein, but should be interpreted in a broad scope consistent with the principles and novel features set forth herein.

The embodiments of the present invention described above are not implemented only through the apparatus and the method, but may also be implemented through a program for realizing a function corresponding to the configuration of the embodiments of the present invention or a recording medium in which the program is recorded. Such a recording medium may be executed not only in a server but also in a user terminal.

Although the embodiments of the present invention have been described in detail above, the scope of the present invention is not limited thereto, and various modifications and improvements of those skilled in the art using the basic concept of the present invention defined in the following claims also fall within the scope of the present invention.

1. A method for predicting an intravitreal injection treatment prognosis, the method comprising:
receiving optical coherence tomography (OCT) images and clinical information of a plurality of diabetic macular edema patients;
extracting one or more pieces of major lesion information from the OCT images;
training a multiple modal deep learning model by inputting the OCT images, the clinical information, and the one or more pieces of major lesion information as inputs and outputting the intravitreal injection treatment prognosis of a diabetic macular edema patient; and
predicting the intravitreal injection treatment prognosis by inputting the OCT images, the clinical information, and the major lesion information of a specific diabetic macular edema patient to the trained multiple modal deep learning model.

2. The method of claim 1, wherein the extracting of the main lesion information comprises: extracting five slices from the OCT image, including two slices on each side of a slice corresponding to the center of the macula; extracting a region of interest (ROI) corresponding to a central region of the macula from the extracted five slices by using a retinal region mask; and extracting the main lesion information from the ROI by using a main lesion mask.

3. The method of claim 1, wherein the major lesion information comprises at least one of a location, a size, a number, or a distribution of a diabetic macular edema lesion.

4. The method of claim 1, wherein the clinical information comprises at least one of age, sex, type of diabetes, glycated hemoglobin level, presence or absence of hypertension, presence or absence of hyperlipidemia, lens condition, vitrectomy history, or previous treatment history of the patient.

5. The method of claim 1, wherein the multi-modal deep learning model is a structure in which a Convolutional Neural Network (CNN) for feature extraction of the OCT image and a Deep Neural Network (DNN) for feature extraction of the clinical information and the main lesion information are combined.

6. The method of claim 5, wherein the predicting of the intravitreal injection treatment prognosis comprises: extracting a first feature by inputting the OCT image to the convolutional neural network (CNN); extracting a second feature by inputting the clinical information and the main lesion information to the deep neural network (DNN); and predicting the intravitreal injection treatment prognosis of the diabetic macular edema patient by combining the first feature and the second feature in a late-fusion manner and inputting the combined feature to a fully-connected network (FCN).

7. The method of claim 1, wherein the predicting of the intravitreal injection treatment prognosis comprises predicting the intravitreal injection treatment prognosis based on a change in central retinal thickness after one or more intravitreal injection treatments.

8. A device for predicting an intravitreal injection treatment prognosis, the device comprising:
a processor including one or more cores; and
a memory, wherein
the processor is configured to:
receive an OCT image and clinical information of a plurality of diabetic macular edema patients;
extract one or more pieces of major lesion information from the OCT image; and
train a multi-modal deep learning model by using the OCT image, the clinical information, and the one or more pieces of major lesion information as inputs, and outputting the intravitreal injection treatment prognosis of a diabetic macular edema patient.

9. A non-transitory computer-readable storage medium storing a computer program comprising instructions for allowing a computer to perform operations comprising:
receiving an OCT image and clinical information of a plurality of diabetic macular edema patients;
extracting one or more pieces of major lesion information from the OCT image;
training a multi-modal deep learning model by inputting the OCT image, the clinical information, and the one or more pieces of major lesion information as inputs and outputting a vitreous intracranial injection treatment prognosis of the diabetic macular edema patients; and
predicting an intravitreal injection treatment prognosis by inputting the OCT image, clinical information, and the major lesion information of a specific diabetic macular edema patient to the trained multi-modal deep learning model.
